(19) United States
(12) Patent Application Publication
(10) Pub. No.: US 2023/0399684 A1
(43) Pub. Date: Dec. 14, 2023

(54) NEW POLYMERASE AND USE THEREOF

(71) Applicant: Genovis AB, Lund (SE)

(72) Inventors: Ola S¿derberg, Osterbybruk (SE); Leonie Wenson, Badenweiler (DE); Bjõrn Hellman, Katrineholm (SE); Erik Bivehed, Uppsala (SE); Johan Heldin, Uppsala (SE)

(21) Appl. No.: 18/034,338

(22) PCT Filed: Oct. 19, 2021
(86) PCT No.: PCT/SE2021/051028

§ 371 Date: Apr. 27, 2023

(30) Foreign Application Priority Data
Oct. 30, 2020 (SE) 2051265-3

(51) Int. Cl. C12Q 1/6844; C12N 9/12

(52) U.S. Cl. CPC C12Q 1/6844; C12N 9/1252; C12Y 207/07007

(57) ABSTRACT

The disclosure relates to a recombinant DNA dependent DNA polymerase having 5′-3′ exonuclease activity and lacking 3′-5′ exonuclease activity, wherein said polymerase is capable of extending DNA polymerisation from a single mismatched base pair and has an error rate of at least 1:1000. The disclosure further relates to a nucleic acid molecule encoding the recombinant DNA dependent DNA polymerase, a method for synthesizing double stranded DNA (dsDNA), a method for obtaining the position of a single strand break in a template dsDNA molecule, a method to introduce mutations in DNA of bacterial or eukaryotic cells, or organisms.

### TECHNICAL FIELD

The present disclosure relates to the field of molecular tools, and in particular to engineered DNA dependent polymerases, useful in biotechnological methods, nucleic acids encoding the same, host cells expressing the same, and various methods utilizing the disclosed molecular tools. The molecular tools find application in DNA analysis, induction of mutagenesis and affinity maturation.

### BACKGROUND ART

The integrity of genomic DNA is a requirement to preserve the genetic information of a cell, and is constantly subjected to threats, due to natural processes, such as replication, transcription and byproducts of metabolic processes, and also from environmental factors such as radiation and chemicals. Failure of a faithful replication and damage repair can lead to mutations in the expressed proteins. Mutations are in general detrimental to the organism but may also, in rare instances, result in changed properties that prove beneficial under certain circumstances.

Methods to detect DNA damage (single-strand and double-strand breaks, or damaged bases) are necessary to determine the consequences of a damaging agent, but also the subsequent repair of such. Examples of current methods for evaluation of the degree of DNA damage per individual cell are the COMET assay (PMID: 6477583) that provides a gross estimate of DNA fragmentation and various sequencing based assays, sequencing-based methods using end-labeling (PMID:27688757) or utilizing DNA alterations in polymerase processivity at modified bases (PMID:31247470) to determine positioning within the genome or to visualize DNA damage in cells by incorporation of fluorophore-labeled nucleotides at the position of the lesion (PMID:29723708).

DNA polymerases have evolved to obtain a high fidelity in order to reduce mutation rate during replication. A disadvantage with the high fidelity is that the DNA polymerases used for replication are not able to bypass damaged bases, e.g. cis-syn thymine dimers, abasic sites and 7,8-dihydro-8-oxoguanine (8-oxoG). There are several translesion DNA polymerases that enable DNA synthesis opposite such damaged bases, that are present in both prokaryotes and eukaryotes to bypass such positions. The translesion DNA polymerases has as a consequence of the more relaxed requirement for identification of nucleotides a higher frequency of miss-incorporation in undamaged DNA. The high error rate of translesion DNA polymerase η, 10⁻² to 10⁻³ (PMID:10601233), introduce mutations in abasic sites generated by the enzyme AID in immunoglobulin genes (PMID:11376341) in B cells, during the process of somatic hypermutation in the development of high affinity antibodies. The processivity of polymerase Tl is however very low, incorporating only a few nucleotides before falling of the DNA strand (PMID:10601233).

The concept of utilizing acquired mutations to evolve recombinant proteins with enhanced affinity have been exploited e.g. utilizing strains of bacteria that has a higher mutation rate (PMID: 9373321, PMID: 8757799). The E. coli strain used in these papers has a defective proofreading ability of DNA polymerase III (PMID: 3054881) that will result in a higher mutation rate during replication.

### SUMMARY

The present inventors have identified a need for an error-prone DNA dependent DNA polymerase that facilitates the improved methods discussed above. Such a DNA dependent DNA polymerase should fulfil the following criteria: (1) it should be highly error prone, (2) it should be able to extend from a mismatched base, (3) it should lack 3′ to 5′exonuclease activity (i.e. no proof-reading) and (4) it should have 5′ to 3′exonuclease activity to remove the strand in front of the nick while it is synthesizing a new strand.

There is also a need for improvement in existing methods to detect DNA damage to increase yield, reduce input requirements and prevent in vitro artifacts, and also to provide possibility for analysis using different platforms.

There is also a need for improved methods for affinity maturation of recombinant proteins, having an inducible error prone DNA polymerase that directs mutations towards specific regions of the DNA. The possibility to make a strain of prokaryotic or eukaryotic cell, carrying a highly error prone polymerase controlled by an inducible promotor, enables control of when mutations are induced and the possibility to expand the bacterial cultures in conditions where only the endogenous polymerases are expressed (i.e. conditions where no additional mutations are inserted). Having a DNA polymerase that initiate DNA synthesis from a DNA nick, degrading the strand in front of it by 5′ to 3′exonuclease activity allows for introduction of short stretches of mutations in regions with DNA nicks.

It is an object of the present invention to provide the error-prone DNA dependent DNA polymerase as discussed above.

It is a further object of the invention to provide a new type of method that will enable the evaluation of not only the degree of DNA damage per individual cell, but also the precise location of these within the genome, which is not possible with current methods.

Thus, in a first aspect the present invention relates to a recombinant DNA dependent DNA polymerase having 5′-3′ exonuclease activity and lacking 3′-5′ exonuclease activity, wherein said polymerase is capable of extending DNA polymerisation from a mismatched base pair and has an error rate of at least 1:1000.

In some embodiments, the recombinant DNA dependent DNA polymerase is a chimeric DNA dependent DNA polymerase, comprising a first domain having 5′-3′ exonuclease activity and lacking 3′-5′ exonuclease activity, and a second domain having capability to extend DNA polymerisation from a mismatched base pair. In some embodiments, the recombinant DNA dependent DNA polymerase comprises a 5′-3′ exonuclease domain of DNA polymerase I and a translesion DNA polymerase f.

In some embodiments, the recombinant DNA dependent DNA polymerase has an amino acid sequence of at least 50%, such as 60%, 70%, 80%, 90%, 95% or 100%, sequence identity to amino acids 15-337 and 350-981 of SEQ ID NO: 2.

The present invention furthermore relates to a nucleic acid molecule encoding the recombinant DNA dependent DNA polymerase according to the invention.

In some embodiments, the nucleic acid molecule has a nucleotide sequence of at least 50%, such as 60%, 70%, 80%, 90%, 95% or 100%, sequence identity to nucleotides 43-1011 and 1048-2943 of SEQ ID NO: 1.

The present invention furthermore relates to a method for synthesizing double stranded DNA (dsDNA) comprising bringing a DNA dependent DNA polymerase according to the invention into contact with a dsDNA template molecule comprising a single strand break, and a reaction mixture comprising three nucleotides selected from dATP, dGTP, dTTP and dCTP, and said reaction mixture not comprising one nucleotide selected from dATP, dGTP, dTTP and dCTP.

In some embodiments, the reaction mixture further comprises dUTP.

In some embodiments, a nucleotide comprised in the reaction is modified, or adapted to be modified, with an affinity ligand.

In some embodiments, the affinity ligand is desthiobiotin.

In some embodiments, the nucleotide modified with an affinity ligand is dUTP.

The present invention furthermore relates to a method for obtaining the position of a single strand break in a template dsDNA molecule, said method comprising
synthesizing dsDNA according to a method according to any one of claims 7-10 to obtain a hybrid dsDNA molecule comprising a first strand originating from the template dsDNA molecule and second strand lacking the nucleotide not comprised in the reaction mixture, in a part of said second strand;
bringing the hybrid dsDNA molecule into contact with a restriction enzyme having a restriction recognition site including the nucleotide lacking from the reaction mixture to cleave the hybrid dsDNA molecule at one or more positions outside the part of the second strand lacking the nucleotide not comprised in the reaction mixture to obtain DNA fragments;
optionally isolating DNA fragments lacking the nucleotide not comprised in the reaction mixture from DNA fragments comprising the nucleotide lacking from the reaction mixture; and
sequencing the DNA fragments not comprising the nucleotide lacking from the reaction mixture;
thereby obtaining the position of the single strand break in the template dsDNA molecule.

In some embodiments, the reaction mixture further comprises a nucleotide modified with an affinity ligand.

In some embodiments, the nucleotide modified with an affinity ligand is not one of dATP, dCTP, dGTP, dTTP.

In some embodiments, the isolation step above is performed by binding the affinity ligand to an affinity binder bound to a solid substrate.

In a further aspect, the present invention relates to a prokaryotic or eukaryotic cell comprising the nucleic acid molecule according to the invention and expressing the encoded DNA dependent DNA polymerase.

In a further aspect, the present invention relates to a method for synthesizing one or more double stranded DNA (dsDNA) molecules comprising bringing a DNA dependent DNA polymerase according to the invention into contact with one or more dsDNA template molecules comprising a single strand break, and a reaction mixture comprising a dsDNA template molecule and four nucleotides selected from dATP, dGTP, dTTP and dCTP.

In a further aspect, the present invention relates to a method for introducing mutations in DNA in a cell, said method comprising expressing the DNA dependent DNA polymerase according to the invention in said cell.

In some embodiments, such methods are non-therapeutic. In some embodiments, the methods are not performed on the human or animal body for therapeutic purposes.

In some embodiments, the methods are performed in vivo in a host cell according to the invention, e.g. the DNA dependent DNA polymerase according to the invention is expressed in order to introduce mutations in the cell's DNA. In some embodiments, the method is performed in vivo in a multi-cellular organism.

In some embodiments, the expression of the DNA dependent DNA polymerase is under control of inducible promotors or tissue specific promotors.

The present disclosure will become apparent from the detailed description given below.

The detailed description and specific examples disclose preferred embodiments of the disclosure by way of illustration only. Those skilled in the art understand from guidance in the detailed description that changes and modifications may be made within the scope of the disclosure.

Hence, it is to be understood that the herein disclosed disclosure is not limited to the particular component parts of the device described or steps of the methods described since such device and method may vary.

### BRIEF DESCRIPTIONS OF THE DRAWINGS

The above objects, as well as additional objects, features and advantages of the present disclosure, will be more fully appreciated by reference to the following illustrative and non-limiting detailed description of example embodiments of the present disclosure, when taken in conjunction with the accompanying drawings.

FIG. 1 provides a schematic overview of method for labeling ssDNA breaks

FIG. 2 A-C illustrate the steps of a method for analyzing the position of a single strand break in a template dsDNA molecule according to the invention. FIG. 2D shows a denaturing PAGE visualize extension of the Hairpin with 4 nucleotides (dATP, dGTP, dTTP and dCTP) or 3 nucleotides (dATP, dGTP and dTTP) at different timepoints, as indicated in figure. FIG. 2E shows a denaturing PAGE stained with Sybrgold (red) and IRDye® 800CW Streptavidin (green) with various ratios of dTTP:desthiobiotin-dUTP.

### DEFINITIONS

It is to be understood that the terminology used herein is for purpose of describing particular embodiments only, and is not intended to be limiting. It should be noted that, as used in the specification and the appended claim, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the words “comprising”, “including”, “containing” and similar wordings does not exclude other elements or steps. All terms are to be given the meaning usually given to them by the person skilled in the art. For the sake of clarity, a few terms are further defined below.

Exonuclease activity—enzymatic activity that work by cleaving nucleotides, one or a few (up to ten) at a time, from the end (exo) of a polynucleotide chain.
Error rate—errors per base per replication cycle. The error rate may be determined as described in the Examples.
Sequence identity—the degree of similarity between two or more nucleotide sequences. The sequence identity between two or more sequences may also be based on alignments using commonly available software for pairwise sequence alignment or multiple sequence alignments available from e.g. the European Bioinformatics Institute (Madeira F, Park Y M. Lee J, et al The EMBL-EBI search and sequence analysis tools APIs in 2019, Nucleic Acids Research. 2019 July; 47(W1):W636-W641).
dsDNA—double-stranded DNA
ssDNA—single-stranded DNA
Affinity ligand—molecules that are capable of binding with very high affinity to either a moiety specific for it or to an antibody raised against it.
Inducible and tissue specific promoters—gene promoters that can be induced under certain conditions, e.g. presence or absence of a biomolecule or chemical, and promoters that only are active in certain cell types or tissues.

### Sequences

The following sequences are relevant to the present disclosure.

In SEQ ID NO: 1 and SEQ ID NO: 2 disclosing one embodiment of the DNA dependent DNA polymerase according to the invention, the 6×His tag with a TEV recognition sequence is highlighted in underline. The 5′-3′exo domain of E-coli DNA polymerase I is highlighted in bold. The 4×TGS spacer is highlighted in Italic. The yeast translesion DNA polymerase η (RAD30), codon usage optimized for expression/translation in E-coli, is highlighted in Italic underline.

Type: DNA
SEQ ID NO: 1
ATGCATCACCATCACCATCACGAAAACCTGTATTTTCAGGGCATGGTTC
AGATCCCCCAAAATCCACTTATCCTTGTAGATGGTTCATCTTATCTTTA
TCGCGCATATCACGCGTTTCCCCCGCTGACTAACAGCGCAGGCGAGCCG
ACCGGTGCGATGTATGGTGTCCTCAACATGCTGCGCAGTCTGATCATGC
AATATAAACCGACGCATGCAGCGGTGGTCTTTGACGCCAAGGGAAAAAC
CTTTCGTGATGAACTGTTTGAACATTACAAATCACATCGCCCGCCAATG
CCGGACGATCTGCGTGCACAAATCGAACCCTTGCACGCGATGGTTAAAG
CGATGGGACTGCCGCTGCTGGCGGTTTCTGGCGTAGAAGCGGACGACGT
TATCGGTACTCTGGCGCGCGAAGCCGAAAAAGCCGGGCGTCCGGTGCTG
ATCAGCACTGGCGATAAAGATATGGCGCAGCTGGTGACGCCAAATATTA
CGCTTATCAATACCATGACGAATACCATCCTCGGACCGGAAGAGGTGGT
GAATAAGTACGGCGTGCCGCCAGAACTGATCATCGATTTCCTGGCGCTG
ATGGGTGACTCCTCTGATAACATTCCTGGCGTACCGGGCGTCGGTGAAA
AAACCGCGCAGGCATTGCTGCAAGGTCTTGGCGGACTGGATACGCTGTA
TGCCGAGCCAGAAAAAATTGCTGGGTTGAGCTTCCGTGGCGCGAAAACA
ATGGCAGCGAAGCTCGAGCAAAACAAAGAAGTTGCTTATCTCTCATACC
AGCTGGCGACGATTAAAACCGACGTTGAACTGGAGCTGACCTGTGAACA
ACTGGAAGTGCAGCAACCGGCAGCGGAAGAGTTGTTGGGGCTGTTCAAA
AAGTATGAGTTCAAACGCTGGACTGCTGATGTCGAAGCGGGCAAATGGT
TACAGGCCAAAGGGGCAAAACCAGCCGCGAAGCCACAGGAAACCAGTGT
TGCAGACGAAGCACCAGAAGTGACGGCAACGACAGGCAGCACCGGGTCG
ACTGGGAGTACGGGTTCCATGTCTAAGTTTACATGGAAAGAGTTAATTC
AATTAGGCAGTCCATCGAAAGCATACGAGTCCTCATTAGCTTGTATCGC
ACATATTGATATGAATGCGTTCTTCGCCCAGGTGGAGCAGATGCGTTGT
GGCCTGTCTAAGGAGGATCCCGTAGTATGCGTTCAGTGGAACAGCATCA
TTGCGGTGTCTTATGCTGCTCGCAAATACGGCATCTCCCGTATGGACAC
CATCCAGGAGGCTCTGAAGAAATGCTCGAACTTAATCCCTATTCATACG
GCCGTCTTCAAGAAAGGAGAAGATTTCTGGCAGTACCATGATGGGTGTG
GGTCGTGGGTACAGGACCCCGCGAAGCAAATCTCGGTCGAGGATCACAA
GGTTTCACTGGAGCCCTATCGTCGTGAATCACGCAAGGCGCTTAAAATC
TTCAAGTCGGCATGCGATTTGGTAGAGCGTGCCTCTATTGACGAGGTAT
TCCTTGACTTGGGACGTATCTGCTTTAACATGTTAATGTTTGACAATGA
GTACGAATTGACAGGGGACTTAAAGTTAAAAGATGCACTGTCTAATATT
CGCGAAGCCTTTATCGGGGGGAATTATGATATTAACTCGCATTTACCGC
TTATTCCTGAGAAAATTAAGAGCTTGAAGTTTGAGGGGGATGTTTTTAA
TCCCGAAGGCCGTGACCTGATCACCGACTGGGACGACGTGATTCTTGCA
CTTGGGAGCCAGGTTTGCAAAGGTATTCGCGACAGTATTAAAGACATCT
TGGGCTATACAACCTCATGCGGGCTTTCATCAACGAAAAACGTCTGTAA
ACTTGCTTCAAACTATAAGAAGCCTGACGCCCAGACTATTGTCAAGAAT
GACTGTCTTCTGGATTTTTTGGACTGCGGAAAGTTCGAGATTACATCCT
TTTGGACGCTGGGTGGAGTCTTGGGAAAGGAACTGATTGATGTCCTTGA
CTTACCTCATGAGAACTCGATCAAACACATTCGTGAGACATGGCCTGAC
AACGCCGGACAGTTGAAGGAGTTTCTGGACGCCAAGGTCAAACAATCTG
ATTATGATCGCTCGACCTCTAACATCGACCCTTTGAAAACCGCTGATCT
GGCCGAAAAGCTTTTTAAACTTTCGCGCGGTCGTTACGGACTTCCATTA
TCTTCACGTCCGGTTGTTAAGTCTATGATGTCCAACAAAAACCTGCGTG
GTAAGTCGTGCAATTCCATCGTTGACTGTATTTCCTGGTTAGAAGTATT
CTGCGCCGAGCTGACATCCCGCATTCAGGATCTTGAACAAGAGTATAAC
AAGATTGTCATCCCTCGTACAGTCTCGATCTCACTGAAAACTAAATCGT
ACGAAGTGTACCGTAAGTCAGGGCCGGTGGCCTACAAGGGCATCAATTT
TCAAAGCCACGAGTTATTGAAAGTCGGGATCAAATTTGTAACCGACCTT
GACATTAAAGGGAAAAATAAATCCTACTATCCGTTAACGAAGCTGTCTA
TGACCATTACTAACTTCGACATCATCGATTTGCAAAAAACTGTTGTGGA
CATGTTTGGGAACCAAGTACACACATTTAAGTCCTCGGCGGGCAAAGAG
GACGAGGAGAAGACAACTAGCAGTAAGGCGGATGAAAAGACACCGAAAC
TGGAATGTTGTAAATATCAAGTGACTTTCACGGACCAAAAAGCACTTCA
AGAGCATGCTGACTACCACCTGGCTTTAAAGCTTTCTGAGGGTCTGAAT
GGAGCGGAGGAGAGCAGTAAAAATTTGTCCTTTGGCGAAAAGCGCTTGC
TGTTCTCTCGTAAACGTCCAAACAGTCAACACACTGCTACTCCCCAGAA
AAAGCAGGTTACTTCATCAAAAAATATTCTTAGTTTCTTCACTCGTAAA
AAATGA
Protein
Theoretical pI: 6.57
Theoretical Mw: 109348.19
SEQ ID NO: 2
MHHHHHHENLYFQGMVQIPQNPLILVDGSSYLYRAYHAFPPLTNSAGEP
TGAMYGVLNMLRSLIMQYKPTHAAVVFDAKGKTFRDELFEHYKSHRPPM
PDDLRAQIEPLHAMVKAMGLPLLAVSGVEADDVIGTLAREAEKAGRPVL
ISTGDKDMAQLVTPNITLINTMTNTILGPEEVVNKYGVPPELIIDFLAL
MGDSSDNIPGVPGVGEKTAQALLQGLGGLDTLYAEPEKIAGLSFRGAKT
MAAKLEQNKEVAYLSYQLATIKTDVELELTCEQLEVQQPAAEELLGLFK
KYEFKRWTADVEAGKWLQAKGAKPAAKPQETSVADEAPEVTATTGSTGS
TGSTGSMSKFTWKELIQLGSPSKAYESSLACIAHIDMNAFFAQVEQMRC
GLSKEDPVVCVQWNSIIAVSYAARKYGISRMDTIQEALKKCSNLIPIHT
AVFKKGEDFWQYHDGCGSWVQDPAKQISVEDHKVSLEPYRRESRKALKI
FKSACDLVERASIDEVELDLGRICENMLMEDNEYELTGDLKLKDALSNI
REAFIGGNYDINSHLPLIPEKIKSLKFEGDVENPEGRDLITDWDDVILA
LGSQVCKGIRDSIKDILGYTTSCGLSSTKNVCKLASNYKKPDAQTIVKN
DCLLDFLDCGKFEITSFWTLGGVLGKELIDVLDLPHENSIKHIRETWPD
NAGQLKEFLDAKVKQSDYDRSTSNIDPLKTADLAEKLFKLSRGRYGLPL
SSRPVVKSMMSNKNLRGKSCNSIVDCISWLEVFCAELTSRIQDLEQEYN
KIVIPRTVSISLKTKSYEVYRKSGPVAYKGINFQSHELLKVGIKFVTDL
DIKGKNKSYYPLTKLSMTITNFDIIDLQKTVVDMFGNQVHTFKSSAGKE
DEEKTTSSKADEKTPKLECCKYQVTFTDQKALQEHADYHLALKLSEGLN
GAEESSKNLSFGEKRLLFSRKRPNSQHTATPQKKQVTSSKNILSFFTRK
K-
SEQ ID NO: 3
CCCAAACCCAATTAATGTACTGCAGAATTCAGCTCGAAGCTTGGCCGGA
TCCAGCGTGGGACTGAGTC
SEQ ID NO: 4
GTCTCGTGTCTGTAAAAACGTACGTAGATGCCATTTCTAAAAAAACAGA
CACGAGACGACTCAGTCCCACGCT
SEQ ID NO: 5
CCCAAACCCAATTAATGTACTGCAGAATTCAGCTCGAAGCTTGGCCGGA
TCCAGCGTGGGACTGAGTCGTCTCGTGTCTGTAAAAACGTACGTAGATG
CCATTTCTAAAAAAACAGACACGAGACGACTCAGTCCCACGCT
SEQ ID NO: 6
CCGGCCAAGCTTCGAGCTGAATTCTGCAGTACATTAATTGGGTTTGGG

### DETAILED DESCRIPTION

The present invention aims to provide a DNA polymerase that is useful in molecular biology, and that exhibit the following features: (1) it is highly error prone, (2) it is able to extend from a mismatched base, (3) it lacks 3′ to 5′exonuclease activity (i.e. no proof-reading) and (4) it has 5′ to 3′exonuclease activity to remove the strand in front of the nick while it is synthesizing a new strand. The present inventors investigated several types of commercially available DNA polymerases and investigated if altered buffer conditions might force them to perform as required, but were unable to achieve the desired features. Hence, the present inventors set out to engineer a DNA polymerase according to the invention that performs in accordance to the specifications above.

In accordance with the above, the present inventors have herein engineered a DNA polymerase, combining an error-prone DNA translesion polymerase with the 5′-3′exonuclease domain of E Coli DNA polymerase I. This chimeric polymerase is capable of replicating DNA, fed only with three nucleotides. The exonuclease activity enables it to initiate replication from a DNA nick and remove the DNA strand in front of the polymerase, to ensure that a long stretch of nucleotides is replaced. We have herein shown that such a chimeric polymerase can be utilized to determine position of ssDNA breaks and will hence provide the research community with a simple way to prepare samples for analysis.

To provide a DNA polymerase with all required featured we engineered a chimeric DNA polymerase by fusing the 5′-3′exonuclease domain of E Coli DNA polymerase I with yeast translesion DNA polymerase η (RAD30). The construction and expression of this chimeric polymerase is described in detail in Example 1.

This chimeric polymerase was confirmed to have the required properties through the experiments as discussed in Example 2.

Thus, in a first aspect, the present invention relates to a recombinant DNA dependent DNA polymerase having 5′-3′ exonuclease activity and lacking 3′-5′ exonuclease activity, wherein said polymerase is capable of extending and/or initiating DNA polymerisation from a single mismatched base pair and has an error rate of at least 1:1000.

In some embodiments, the recombinant DNA dependent DNA polymerase is a chimeric DNA dependent DNA polymerase, comprising a first domain having 5′-3′ exonuclease activity and lacking 3′-5′ exonuclease activity, and a second domain having capability to extend DNA polymerisation from a mismatched base pair.

In some embodiments, the first domain derives from the 5′-3′ exonuclease domain of e.g. DNA polymerase I, T7 DNA polymerase, Polymerase γ, Polymerase θ, Polymerase v, Exonuclease II or Flap structure-specific endonuclease 1.

The 5′-3′ exonuclease activity may thus be conferred to the DNA dependent DNA polymerase through inclusion of a nucleic acid encoding a protein domain conferring 5′-3′ exonuclease activity to an existing enzyme into a recombinant nucleic acid encoding the DNA dependent DNA polymerase according to the invention. Such protein domains, and nucleic acids encoding them, are known in the art. In some embodiments, the protein domain included in the DNA dependent DNA polymerase according to the invention derives from the 5′-3′ exonuclease domain of e.g. DNA polymerase I, T7 DNA polymerase, Polymerase γ, Polymerase θ, Polymerase v, Exonuclease II or Flap structure-specific endonuclease 1.

In some embodiments, the polymerase originates from a prokaryotic or a eukaryotic organisms, such as bacteria, yeasts, fungi, vertebrates, and mammals. In some embodiments, the polymerase originates from E. coli, S. cervisiae, or any other model organism.

In one embodiment, the 5′-3′ exonuclease activity is conferred to the DNA dependent DNA polymerase through inclusion of a nucleic acid encoding a protein domain conferring 5′-3′ exonuclease activity to DNA polymerase I. In one embodiment, the DNA polymerase I originates from E. coli. In one embodiment, the 5′-3′ exonuclease activity is conferred to the DNA dependent DNA polymerase through inclusion of a nucleic acid having a nucleotide sequence of at least 50%, such as 60%, 70%, 80%, 90%, 95% or 100%, sequence identity to nucleotides 43-1011 of SEQ ID NO: 1. In one embodiment, the 5′-3′ exonuclease activity is conferred to the DNA dependent DNA polymerase through inclusion of a nucleic acid encoding an amino acid sequence having at least 50%, such as 60%, 70%, 80%, 90%, 95% or 100%, sequence identity to amino acids 15-337 of SEQ ID NO: 2.

In some embodiments, the second domain derives from a translesion DNA polymerase.

The DNA polymerase activity may be conferred to the DNA dependent DNA polymerase through inclusion of a nucleic acid encoding a protein domain conferring suitable DNA polymerase activity to an existing enzyme into a recombinant nucleic acid encoding the DNA dependent DNA polymerase according to the invention, to confer an error rate of at least 1:1000 to the resulting DNA dependent DNA polymerase. Such protein domains, and nucleic acids encoding them, are known in the art. In some embodiments, the protein domain included in the DNA dependent DNA polymerase according to the invention derives from DNA Polymerase ι, DNA Polymerase κ, DNA Polymerase η, DNA Polymerase ζ, DNA polymerase IV or DNA polymerase V. In some embodiments, the polymerase originates from E. coli, S. cervisiae, or any other model organism.

In one embodiment, the DNA polymerase activity is conferred to the DNA dependent DNA polymerase through inclusion of a nucleic acid encoding a translesion DNA polymerase η. In one embodiment, the translesion DNA polymerase η originates from S. cerevisiae. In one embodiment, the DNA polymerase activity is conferred to the DNA dependent DNA polymerase through inclusion of a nucleic acid having a nucleotide sequence of at least 50%, such as 60%, 70%, 80%, 90%, 95% or 100%, sequence identity to nucleotides 1048-2943 of SEQ ID NO: 1. In one embodiment, the DNA polymerase activity is conferred to the DNA dependent DNA polymerase through inclusion of a nucleic acid having a nucleotide sequence encoding an amino acid sequence having at least 50%, such as 60%, 70%, 80%, 90%, 95% or 100%, sequence identity to amino acids 350-981 of SEQ ID NO: 2.

In a further aspect, the present invention relates to a method for synthesizing double stranded DNA (dsDNA) comprising bringing a DNA dependent DNA polymerase according to the invention into contact with a dsDNA template molecule comprising a single strand break, and a reaction mixture comprising three nucleotides selected from dATP, dGTP, dTTP and dCTP, and said reaction mixture not comprising one nucleotide selected from dATP, dGTP, dTTP and dCTP. In one embodiment, the reaction mixture does not comprise dATP. In one embodiment, the reaction mixture does not comprise dGTP. In one embodiment, the reaction mixture does not comprise dTTP. In one embodiment, the reaction mixture does not comprise dCTP. In one embodiment the reaction mixture does comprise dUTP. Exemplary conditions for performing the method are as set out in Example 2.

In one embodiment, the reaction mixture further comprises a nucleotide modified with an affinity ligand. Providing a nucleotide carrying an affinity ligand in the reaction mixture leads to its incorporation in the newly synthesized DNA strand, and facilitates easy extraction of the newly synthesized DNA molecules from the reaction by e.g. affinity chromatography or affinity binding to a solid substrate. Affinity ligands along with corresponding affinity binders are well-known in the art. Examples include biotin or desthiobiotin together with streptavidin or avidin, digoxigenin (DIG)/anti-DIG-antibody and dinitrophenol (DNP)/anti-DNP-antibody, fluorophores (e.g. fluorescein) and anti-fluorophore-antibody (e.g. anti-fluorescein-antibody) The nucleotide may also be adapted to be modified with an affinity ligand, such as be incorporating a 5-ethynyl-group that can subsequently be used to couple an affinity ligand to the newly synthesized DNA molecule. In one embodiment the affinity binder is desthiobiotin.

In one embodiment, the nucleotide modified with an affinity ligand is desthiobiotinylated dUTP. In one embodiment the affinity binder is desthiobiotin. In one embodiment, the nucleotide modified with an affinity ligand is desthiobiotinylated dATP.

The present invention further aims to facilitate and provide a method to specifically label positions in genomic DNA that has been subjected to single strand breaks, which method uses an error-prone DNA polymerase and only three nucleotides. By removing e.g. dCTP from the dNTPs (i.e. only providing dATP, dGTP, dTTP) added to the reaction, all cytidines would be depleted from the newly synthesized strand. As this will cause mismatches in the synthesized regions, it will destroy the recognition sites for restriction enzymes that will only be able to cut the DNA outside these regions.

Thus, in one aspect, the invention relates to a method for obtaining the position of a single strand break in a template dsDNA molecule, said method comprising
synthesizing dsDNA according to a method according to the above to obtain a hybrid dsDNA molecule comprising a first strand originating from the template dsDNA molecule and second strand lacking the nucleotide not comprised in the reaction mixture, in a part of said second strand;
bringing the hybrid dsDNA molecule into contact with a restriction enzyme having a restriction recognition site including the nucleotide lacking from the reaction mixture to cleave the hybrid dsDNA molecule at one or more positions outside the part of the second strand lacking the nucleotide not comprised in the reaction mixture to obtain DNA fragments;
optionally isolating DNA fragments lacking the nucleotide not comprised in the reaction mixture from DNA fragments comprising the nucleotide lacking from the reaction mixture; and
sequencing the DNA fragments not comprising the nucleotide lacking from the reaction mixture;
thereby obtaining the position of the single strand break in the template dsDNA molecule.

The position of the single strand break in the template dsDNA molecule is obtained by identifying the point where one nucleotide is depleted from the sequence, e.g. if dATP is removed from the dNTP mixture the position in the sequence where all adenosines are replaced with guanosine, thymidine or cytidine. The sequence upstream the lesion will contain all four nucleotides, but from the nick one of the nucleotides will be replaced with an erroneous one.

The sequence in the downstream region, where the nucleotides have been replaced, can be identified by sequencing both strands, or by comparing the sequence of the newly synthesized strand with a reference sequence. The downstream region together with the upstream region will identify the genomic location.

In one embodiment, the reaction mixture further comprises a nucleotide modified with an affinity ligand. This facilitates the isolation of newly synthesized DNA molecules by use of affinity binders specifically binding the affinity ligand, wherein the affinity binder preferably is bound to a solid substrate. Affinity ligands are further described above. In some embodiments, the nucleotide modified with an affinity ligand is not one of dATP, dCTP, dGTP, dTTP. In some embodiments, the nucleotide modified with an affinity ligand is dUTP. Affinity ligands, such as biotin, can be used to extract the fraction of the reaction mix in which the DNA polymerase has inserted new nucleotides. As only a very small fraction of the DNA may contain DNA nicks, affinity-based purification methods using e.g. streptavidin to pull down biotinylated molecules will increase the fraction of molecules of interest. Hence, reducing costs (i.e. not generate sequencing reads of non-modified DNA).

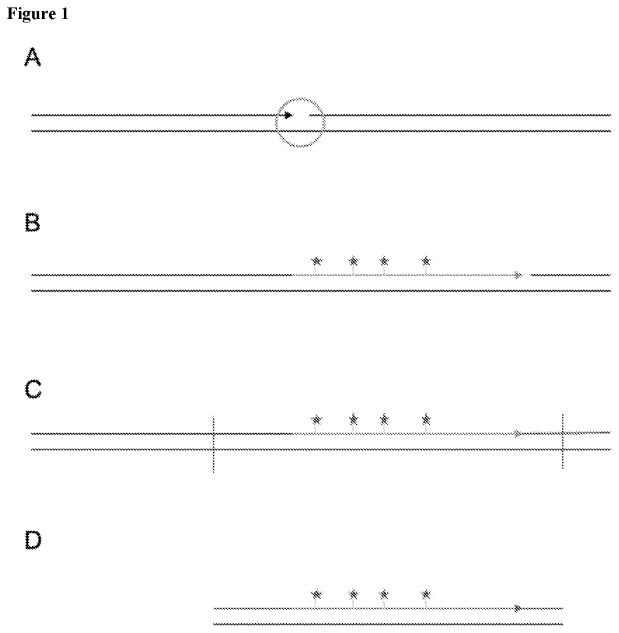

FIG. 1 provides a schematic overview of method for labeling ssDNA breaks. In order to be able to extract the DNA fragments produced by the restriction enzymes, the dNTP reaction mix used for the synthesis may contain a pool of modified nucleotides that could be used for pull down, e.g. desthiobiotinylated dUTP.

Initially (FIG. 1A) a spontaneous ssDNA break is formed, or created to remove modified bases or abasic sites by e.g. UDG, FPG: T4 PDG or Endo VIII.

Then an error-prone DNA polymerase according to the invention binds to a nick in DNA and degrades the downstream region through its 5′-3′exonuclease activity and incorporates new nucleotides (FIG. 1B). Stars indicate incorporated modified nucleotide (e.g. desthiobiotin-dUTP). In addition, only three nucleotides are used (dATP, dGTP, dTTP), hence the polymerase replaces all dC with dT (or desthiobiotin-dU).

A restriction enzyme then cleaves the DNA outside the polymerized area (to the left and right of the vertical bars) as the enzyme require dC for recognition (FIG. 1C). The desthiobiotinylated DNA fragments are bound by streptavidin-coated beads for purification of the DNA fragments that have been modified by the error-prone DNA polymerase (FIG. 1D)

Adaptors are ligated for downstream sequencing. The position of the DNA nicks, that were used to prime DNA synthesis, are determined by position of first misincorporated base.

In one aspect, the present invention relates to a host cell comprising a nucleic acid molecule encoding the DNA dependent DNA polymerase according to the invention. Such a host cell may be prokaryotic, such as a bacteria, e.g. Escherichia coli, Lactobacillus reuteri, other Lactobacilli, Bacillus spp. The host cell, or organism, may also be eukaryotic, such as a yeast, e.g. Saccharomyces cerevisiae, Pichia pastoris, or Schizosaccharomyces pombe, or fungi, e.g. Aspergillus oryzae, mammalian cell e.g. Homo sapiens, Mus musculus, Rattus norvegicus or plants e.g. Arabidopsis thaliana. The nucleic acid molecule may be codon-optimized for the particular species of host cell to be used. The nucleic acid may be operably linked to a constitutive, inducible, or tissue-specific promoter to ensure regulated expression under certain growth conditions, upon stimuli or in defined populations of cells or tissues. This enables controlled increment of mutation rates in defined cell population and for defined periods of time. Hence, it can be used as model systems for several applications in medical research e.g. as a model for cancer development and progression, development of resistance to targeted therapy and as a model system to determine how mutational burden in malignant cells influences the immune response.

The chimeric polymerase according to the invention can also be used to provide an alternative approach to generate mutations in vivo, e.g. in a host cell as described above. The chimeric polymerase according to the invention hence provides an alternative approach to generate mutations in vivo and can be utilized to increase mutation rate in a controlled manner, in any cell type. This can be applied to increase affinity of recombinant affinity reagents, produced in bacterial or eukaryotic cells. Selection of increased affinity could be performed while the cells are induced to mutate, i.e. they would compete for binding to the antigen on a substrate. Such system would provide affinity maturation of recombinant affinity reagent, or any protein of interest, in any cell type.

Having confirmed that a chimeric polymerase according to the invention fulfills all requirements for a DNA polymerase to be used for detection of ssDNA breaks as described above, we further analyzed if it would be able to modify the nucleotide sequence in a nicked plasmid. In order to selectively introduce nicks, we utilized the Nickase Nt.BsmAI that will generate six nicks in a pcDNA3.1 plasmid. The nicked plasmid was treated with the chimeric polymerase obtained in Example 1 with three nucleotides for 15 minutes, and subsequently digested with the restriction enzyme RsaI. Adaptors were ligated to the ends and amplified by PCR. The PCR amplicons were cloned into TOPO-vectors and transformed into E coli. Twenty single colonies were picked and sequenced and nucleotide exchange downstream the nick site was detected in three of these colonies (results shown in FIG. 3). The data confirms that a chimeric polymerase according to the invention can initiate replication from a nicked DNA molecule and synthesize at least 40 nucleotides during a 15 minutes incubation.

Thus, in a further aspect, the present invention relates to a method for synthesizing one or more double stranded DNA (dsDNA) molecules comprising bringing a DNA dependent DNA polymerase according to the invention into contact with one or more dsDNA template molecules comprising a single strand break, and a reaction mixture comprising a dsDNA template molecule and four nucleotides selected from dATP, dGTP, dTTP and dCTP.

In one embodiment, the method according to this aspect is performed in vivo in a host cell according to the invention.

In performing the invention as described herein, the skilled person may use common general knowledge in the art. Such knowledge is available in e.g. Molecular Cloning: A Laboratory Manual, 2nd edition (1989) (Sambrook, Fritsch, and Maniatis); Molecular Cloning: A Laboratory Manual, 4th edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2nd edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton e/a/., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

All references cited herein are expressly incorporated by reference.

The following examples are provided to further illustrate the invention. While being illustrative and informative, they are not limiting of the invention, which is as defined above and in the appended claims.

### Example 1: Expression of Recombinant Chimeric Polymerase

The polymerase (SEQ ID NO: 1) was designed by fusing the 5′-3′exonuclease domain of E Coli DNA polymerase I (the first 969 nucleotides) to 5′-end of yeast RAD30 (with codon optimization for expression in E coli). A short spacer (4×TGS) was included between them and a 6×His tag with a TEV recognition sequence was placed 5′ of the construct to allow for purification of the recombinant protein. The construct was placed in a pBAD vector. Vectors for the different DNA polymerases were transformed into E. coli (LMG194) and were expanded in LB medium containing ampicillin at 37° C. and vigorous agitation, first as an overnight culture and thereafter transferred to a larger production culture. The production culture was cultured until OD₆₀₀=0.5 was reached. Thereafter a final concentration of 0.02% L-arabinose was added and the bacteria incubated at RT overnight during vigorous agitation. The bacteria were harvested by centrifugation for 15 minutes at 6000×g and 4° C., and lysed for 30 min at 4° C. with binding buffer (50 mM sodium phosphate, 500 mM NaCl, pH 7.4) supplemented with 0.2 mg/ml Lysozyme, 1 mM MgCl₂, 0.25% Triton-x and 1× cOmplete protease inhibitor without EDTA (4693159001, vwr). The lysate was cleared by centrifugation for 15 min at 13000×g at 4° C. and thereafter the lysate was passed through a 0.45 μm filter. A His GraviTrap™ TALON® column (29000594, Fisher scientific) was equilibrated with binding buffer according to manufacturer's recommendations and thereafter the lysate was applied to the column at 4° C. After the lysate had passed through the column it was washed two times with 10 mL binding buffer supplemented with 5 mM Imidazole at 4° C. The His-tagged polymerase was eluted using binding buffer supplemented with 50 mM Imidazole at 4° C. The eluate was concentrated and the buffer changed to 2× storage buffer (50 mM Tris·HCl, 2 mM DTT, 0.2 mM EDTA and pH 7.4 at 25° C.) using Amicon 10 kDa spin filterer. The enzyme concentration was measured using a Nanodrop and thereafter a final concentration of 50% Glycerol was added.

### Example 2: Confirmation of Polymerase Properties

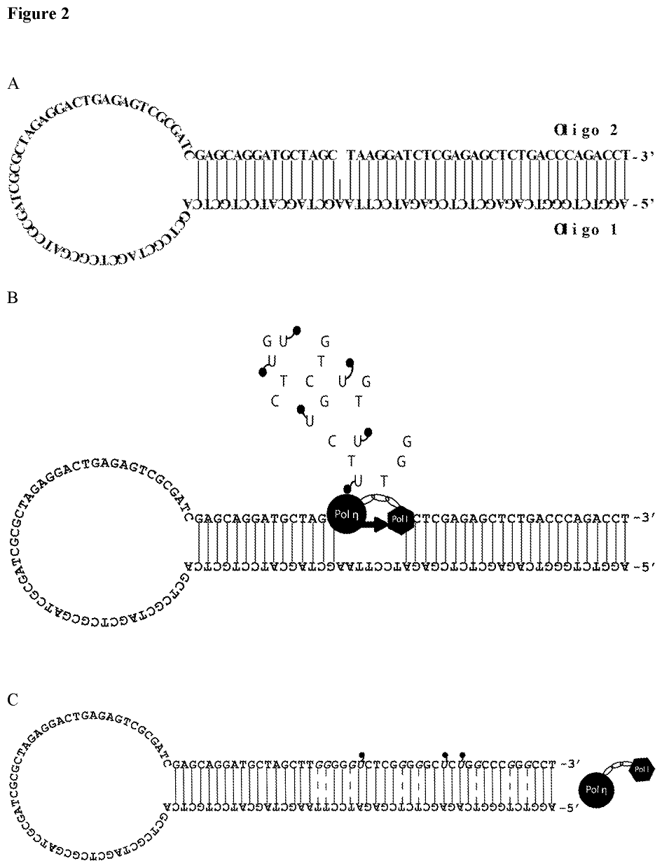

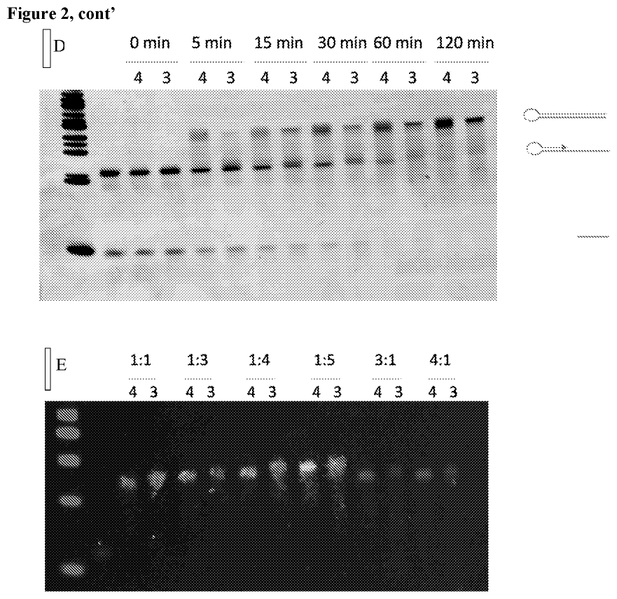

Two oligos: 5′-CCCAAACCCAATTAATGTACTGCAGAATTCAGCTCGAAGCTT GGCCGGATCCAGCGTGGGACTGAGTC (SEQ ID NO: 3) and Phosphate-5′-GTCTCGTGTCTGTAAAAAC GTACGTAGATGCCATTTCTAAAAAAACAGACACGAGACGACTCAGTCCCACGCT (SEQ ID NO: 4) (20 uM of each) were ligated over night at 4° C. with T4 ligase (EL0011, Thermo Scientific) in ligase reaction buffer (50 mM Tris-HCl, 10 mM MgCl₂, 1 mM DTT, 1 mM ATP and pH 7.6 at 25° C.) to form hairpin-shaped DNA fragments (SEQ ID NO: 5) with overhangs of 51 bases (FIG. 2 A). To extend the overhang and create a blunt-ended hairpin, the DNA fragments with a final concentration of 0.02 uM, were mixed with an oligonucleotide 5′-CCGGCCAAGC TTCGAGCTGAATTCTGCAGTACATTAATTGGGTTTGGG (SEQ ID NO: 6) that hybridize to the hairpin (FIG. 2B) and were incubated in 1×NEBuffer™2 (B7002S, New England Biolabs, 50 mM NaCl, 10 mM Tris-HCl, 10 mM MgCl₂, 1 mM DTT and pH 7.9 at 25° C.) supplemented with 0.1 mM MnCl₂. 0.05 uM of the Sloppymerase and either four nucleotides i.e. dATP, dCTP, dGTP and dTTP with a final concentration of 0.1 mM respectively or three nucleotides i.e. dATP (0.1 mM), dGTP (0.1 mM) and dTTP (0.2 mM) were added to the reaction. For the introduction of biotinylated nucleotides, 0.1 mM of the dTTP were supplemented for 0.1 mM of Desthiobiotin-X-(5-aminoallyl)-dUTP. The samples were then incubated at 37° C. for 120 min. The enzyme was heat inactivated at 75° C. for 20 min.

The samples were run with denaturing polyacrylamide gel electrophoresis (PAGE). To visualize the DNA, the gel was stained with 1×SYBR™ Gold Nucleic Acid Gel Stain (S11494, Thermo Fisher Scientific). In addition, to visualize the incorporation of desthiobiotinylated nucleotides the gel was stained with IRDye® 800CW Streptavidin (926-32230, LI-COR Biosciences) in a final concentration of 0.2 ug/ml.

The Sloppymerase treated samples were amplified with PCR using Phusion™ High-Fidelity DNA Polymerase according to the manual. The blunt-ended PCR products were purified with an ethanol precipitation. The purified PCR products were then cloned into a plasmid vector using Zero Blunt® TOPO® PCR cloning (450245, Thermo Fisher Scientific). The manufacturer's recommendations were followed for cloning and subsequent transformation of One Shot™ TOP10 Chemically competent E. coli (C404010). After isolation of the plasmid DNA using PureLink® Quick Plasmid Miniprep Kit (K210011, Thermo Fisher Scientific) the samples were sent to Eurofins Genomics to be sequenced.

The error rate was determined by extending a DNA hairpin, designed to have a 5′-overhang. The hairpin was then extended by the DNA polymerase according to the invention, either using four nucleotides (dCTP, dGTP, dATP, dTTP) or with three nucleotides (dCTP, dGTP, dTTP), i.e. omitting dATP. The extension was confirmed on a denaturing PAGE and adapters were ligated on the extended hairpins. The products were then sent for DNA sequencing, utilizing the primer site in the adaptor and a primer site in the loop of the hairpin for amplification and sequencing. The reads were analysed to determine frequency of misincorporated nucleotides, deletions and insertions. The sequence of the DNA hairpin is set by the oligodesign, but to control for errors introduced by DNA synthesis extension of the hairpin by a proof-reading DNA polymerase with low error rate (Phusion DNA polymerase) was used as a comparison. Frequencies of misincorporations, deletions and insertions, above what was determined for Phusion DNA polymerase was considered as true errors and used to determine the error rate for the DNA polymerase according to the invention, the frequency of errors made in the extended hairpin.

In order to determine if the chimeric polymerase could replicate DNA when only three nucleotides were provided, we designed the above described DNA hairpin of 142 nucleotides (SEQ ID NO: 5) with a 5′-overhang of 52 nucleotides (“Oligo 1” FIG. 2A). The overhang comprises recognition sites for several restriction enzymes. To monitor the 5′-3′exonuclease activity, we hybridized the hairpin to an oligonucleotide with 49 nucleotides (SEQ ID NO: 6) complementary to the hairpin, creating a gap of 3 nucleotides (“Oligo 2” FIG. 2A). As the primer and template is linked together, replication will be measured as an increase in length (i.e. from 142 to 194 nucleotides) on a denaturing PAGE. Disappearance of the band at the size of 49 nucleotides will show exonuclease activity of the chimeric polymerase. When provided with all four dNTPs the chimeric polymerase will extend the hairpin with the correct nucleotides and when one nucleotide is removed from the mixture, e.g. dATP (FIG. 2B), several mismatches will be created (FIG. 2C). Such mismatches will destroy the recognition sites for restriction enzymes and will hence result in that only regions outside the synthesized region will be cleaved. Inclusion of a nucleotide modified with an affinity ligand, such as desthiobiotinulated dUTP, results in incorporation of the affinity ligand in the newly synthesized dsDNA, facilitating affinity separation.

The chimeric polymerase was incubated with the oligonucleotide system described above, together with three nucleotides (dATP, dGTP and dTTP) or four nucleotides and the reactions were stopped at different timepoints (5, 15, 30, 60 and 120 minutes). The samples were then run on a denaturing PAGE and the amplification was determined as an increase in size of the hairpin, and the exonuclease activity was determined by degradation of the hybridized oligonucleotide (FIG. 2D). The data clearly shows that the chimeric polymerase has polymerization activity as well as exonuclease activity and also a fidelity low enough to allow for polymerization in the presence of only three nucleotides, albeit at a decreased speed. Sequencing of the extended hairpin shows that omitting dCTP from the reaction mix generate a product devoid of C (Table 1, −dCTP) in contrast to when all four nucleotides are present in the reaction mix (Table 1, dNTP). Misincorporated nucleotides are shown in bold letters, and the positions where C should have been incorporated, i.e. positions where G is positioned in the complementary DNA strand, are shown as shaded columns (indicated with arrows).

TABLE 1

| | |
|---|---|
| | 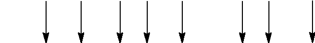 |
| Oligo -dCTP 1. F 2. F 3. F 4. F 5. F 6. F 7. F 8. F 9. F 10. F 11. F 12. F | 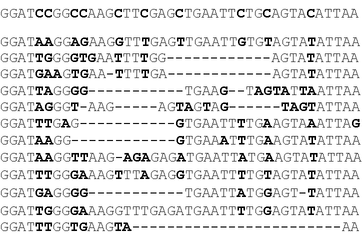 |
| dNTP | |
| 13. F | GGATCCGGCCAAGCTTCAAGCTGAATTCTGCAGTACATTAA |
| 14. F | GGATCCG----AGCGTCGAGCTGAATTCTGCAGTCCATTAT |
| 15. F | GGATCCGGCCAAGCTTCGAGCTGAATCCTGCAGTACATTAA |
| 16. F | GGATCCGGCCAAGCTTCAAGCTGAATTCTGCAGTACATTAA |
| 17. F | GGATCCGGCCAAGCTTCGAGCTGAATTCTGCAGTACATTAG |
| 18. F | GGATCCGGCCAAGCTTCGAGCTGAATTCTGCAGTACATTAA |
| 19. F | GGATCCGGCCAAGTTTCGAGCTGAATTCTGCAGTACATTAA |
| 20. F | GGATCCGGCCAAGCTTCGAGCTGAATTCTGCAGTACATTAA |

To confirm that the chimeric polymerase is able to incorporate desthiobiotin-dUTP, we added the desthiobiotin-dUTP at various ratios, towards dTTP, and performed an experiment with either three or four nucleotides present (i.e. without or with dCTP). The PAGE gel was stained with Sybrgold to visualize DNA and IRDye® 800CW Streptavidin to visualize incorporated desthiobiotin-dUTP and showing that the chimeric polymerase successfully incorporates also modified dNTPs (FIG. 2E).

1. A recombinant DNA dependent DNA polymerase having 5′-3′ exonuclease activity and lacking 3′-5′ exonuclease activity, wherein said polymerase is capable of extending DNA polymerisation from a mismatched base pair and has an error rate of at least 1:1000.

2. The recombinant DNA dependent DNA polymerase according to claim 1, being a chimeric DNA dependent DNA polymerase, comprising a first domain having 5′-3′ exonuclease activity and lacking 3′-5′ exonuclease activity, and a second domain having capability to extend DNA polymerisation from a mismatched base pair.

3. The recombinant DNA dependent DNA polymerase according to claim 2, wherein the first domain is a 5′-3′ exonuclease domain of DNA polymerase I and the second domain is a translesion DNA polymerase η.

4. The recombinant DNA dependent DNA polymerase according to any one of claims 1-3, having an amino acid sequence of at least 50%, such as 60%, 70%, 80%, 90%, 95% or 100%, sequence identity to amino acids 15-337 and 350-981 of SEQ ID NO: 2.

5. A nucleic acid molecule encoding the recombinant DNA dependent DNA polymerase according to any one of claims 1-4.

6. The nucleic acid molecule according to claim 5, having a nucleotide sequence of at least 50%, such as 60%, 70%, 80%, 90%, 95% or 100%, sequence identity to nucleotides 43-1011 and 1048-2943 of SEQ ID NO: 1.

7. A method for synthesizing double stranded DNA (dsDNA) comprising bringing a DNA dependent DNA polymerase according to any one of claims 1-5 into contact with a dsDNA template molecule comprising a single strand break, and a reaction mixture comprising three nucleotides selected from dATP, dGTP, dTTP and dCTP, and said reaction mixture not comprising one nucleotide selected from dATP, dGTP, dTTP and dCTP.

8. The method for synthesizing dsDNA according to claim 7, wherein the reaction mixture further comprises dUTP.

9. The method for synthesizing dsDNA according to any one of claim 7 or 8, wherein a nucleotide comprised in the reaction is modified, or adapted to be modified, with an affinity ligand.

10. The method for synthesizing dsDNA according to claim 9, wherein the affinity ligand is desthiobiotin.

11. The method for synthesizing dsDNA according to claim 9 or 10, wherein the nucleotide modified with an affinity ligand is dUTP.

12. A method for obtaining the position of a single strand break in a template dsDNA molecule, said method comprising
synthesizing dsDNA according to a method according to any one of claims 7-11 to obtain a hybrid dsDNA molecule comprising a first strand originating from the template dsDNA molecule and second strand lacking the nucleotide not comprised in the reaction mixture, in a part of said second strand;
bringing the hybrid dsDNA molecule into contact with a restriction enzyme having a restriction recognition site including the nucleotide lacking from the reaction mixture to cleave the hybrid dsDNA molecule at one or more positions outside the part of the second strand lacking the nucleotide not comprised in the reaction mixture to obtain DNA fragments;
optionally isolating DNA fragments lacking the nucleotide not comprised in the reaction mixture from DNA fragments comprising the nucleotide lacking from the reaction mixture; and
sequencing the DNA fragments not comprising the nucleotide lacking from the reaction mixture;
thereby obtaining the position of the single strand break in the template dsDNA molecule.

13. The method according to claim 12, wherein the reaction mixture further comprises a nucleotide modified with an affinity ligand.

14. The method according to claim 13, wherein the nucleotide modified with an affinity ligand is not one of dATP, dCTP, dGTP, dTTP.

15. The method according to any one of claim 13 or 14, wherein the isolation step of claim 12 is performed by binding the affinity ligand to an affinity binder bound to a solid substrate.

16. A prokaryotic or eukaryotic cell comprising the nucleic acid molecule according to claim 5 or 6 and expressing the encoded DNA dependent DNA polymerase.

17. A method for introducing mutations in DNA in a cell, said method comprising expressing the DNA dependent DNA polymerase according to any one of claims 1-4 in said cell.

18. The method according to claim 17, wherein the method is performed in vivo in a cell according to claim 16.

19. The method according to claim 18, wherein the method is performed in vivo in a multi-cellular organism.

20. The method according to any one of claims 17-19, wherein expression of the DNA dependent DNA polymerase is under control of inducible promotors or tissue specific promotors.
